(19) United States
(12) Patent Application Publication
(10) Pub. No.: US 2026/0060700 A1
(43) Pub. Date: Mar. 5, 2026

(54) SHEATHED SCORING ELEMENTS

(71) Applicant: BOSTON SCIENTIFIC SCIMED, INC., Maple Grove, MN (US)

(72) Inventors: John Kilcooley, Gort (IE); Michelle Hannon, Galway (IE)

(73) Assignee: BOSTON SCIENTIFIC SCIMED, INC., Maple Grove, MN (US)

(21) Appl. No.: 19/317,421

(22) Filed: Sep. 3, 2025

Related U.S. Application Data
(60) Provisional application No. 63/690,547, filed on Sep. 4, 2024.

(51) Int. Cl. A61B 17/22; A61B 17/00; A61B 17/32; A61M 25/10

(52) U.S. Cl. CPC A61B 17/22; A61B 17/00234; A61B 17/32; A61M 25/104; A61B 2017/00238; A61B 2017/00305; A61B 2017/00557; A61B 2017/22001; A61B 2017/22069; A61M 2210/12

(57) ABSTRACT

An example system for treatment of a vessel lesion, the system comprising a catheter shaft; a balloon having an exterior surface, where the balloon is configured to move between a deflated condition and an inflated condition; and a sheathed scoring element that is separate from the balloon and includes: a scoring member having a proximal end region, a distal end region, and a body portion therebetween; and a sheath including a channel, where the sheath is configured to move relative to the scoring member between: a delivery condition where at least a portion of the scoring member is located in the channel; and a scoring condition where the portion of the scoring member is located distal to a distal end of the channel and is configured to contact a target site.

### CROSS REFERENCE TO RELATED APPLICATIONS

The present application claims the benefit of U.S. Provisional Patent Application Ser. No. 63/690,547, filed Sep. 4, 2024, the disclosure of which is incorporated herein by reference.

### TECHNICAL FIELD

The disclosure pertains to angioplasty balloon catheters. More particularly, the disclosure pertains to systems and apparatuses including a balloon and a separate sheathed scoring element that includes a sheath and a scoring member.

### BACKGROUND

Arterial blockages, which are also called stenosis, lesions, stenotic lesions, etc., are typically caused by the build-up of atherosclerotic plaque on the inside wall of an artery. In fact, several such stenoses may occur contiguously within a single artery. This can result in a partial, or even complete, blockage of the artery. As a result of the danger associated with such a blockage, several methods and procedures have been developed to treat stenoses. One such method is an angioplasty procedure which uses an inflatable balloon to dilate the blocked artery.

Angioplasty balloons have enjoyed widespread acceptance in the treatment of stenoses. The efficacy of the dilation of a stenosis may be enhanced by first, or simultaneously, incising the material that is creating the stenosis. Consequently, developments have been made to equip angioplasty balloons with cutting edges, or atherotomes, which are intended to incise a stenosis during the dilation procedure. For example, inflatable angioplasty medical cutting balloons having a number of atherotomes mounted longitudinally on the surface of the balloon may be employed. Upon inflation of the medical cutting balloon, the atherotomes induce a series of longitudinal cuts into the surface of the stenotic material as the balloon expands to dilate the stenosis. As a result of such cuts, the stenosis is more easily dilated, and the likelihood of damaging the artery during dilation is reduced. If a stent is required, the risk of stent under expansion is reduced if these balloons are used for lesion preparation, as arterial plaque is modified and calcified lesions are disrupted.

Blades in many existing cutting balloon assemblies tend to be fairly rigid, particularly in the axial direction. The rigid axial structure of the blade naturally limits the ability of the blades to elongate with the underlying balloon material during balloon expansion. Existing blades (e.g., fixed blades which are coupled to a surface of a balloon) also tend to be fairly rigid in the transverse direction as well. This limits the flexibility of the balloon as it is advanced through the tortuous confines of a vessel or other body lumen. Moreover, the blades or other cutting elements may be prone to inadvertent contact of tissue and/or entanglement, particularly during navigation of the cutting balloon to a target site within a vessel. For instance, the blades or other cutting elements may be affixed to an exterior surface (e.g., permanently attached to the exterior surface of the balloon) and thus may contact tissue that is adjacent to the cutting balloon. Such inadvertent contact may cause undue tissue damage (e.g., at locations along a vessel other than at a target site), may entangle the cutting elements (e.g., may entangle cutting wires), and/or otherwise may lead to issues. For instance, physicians may have concerns with tracking or navigating bulky drug coated balloons, such as those with one or more cutting blades or other cutting elements (e.g., with cutting wires permanently coupled to a surface of a balloon) through a vessel, particularly from a safety standpoint. For instance, atherotomes or blades, such as those that are several millimeters in length, may have difficulties navigating along vasculatures, such as difficulty navigating bends along a vessel. There is an ongoing need for improved cutting or scoring apparatus and systems such as those that are suitable for angioplasty or other medical procedures.

### SUMMARY

In some aspects, the present disclosure pertains to a system for treatment of a vessel lesion, the system comprising: a catheter shaft; a balloon having an exterior surface, wherein the balloon is configured to move between a deflated condition and an inflated condition; and a sheathed scoring element that is separate from the balloon and includes: a scoring member having a proximal end region, a distal end region, and a body portion therebetween; and a sheath including a channel, wherein the sheath is configured to move relative to the scoring member between: a delivery condition where at least a portion of the scoring member is located in the channel; and a scoring condition where the portion of the scoring member is located distal to a distal end of the channel and is configured to contact a target site.

In some aspects, which can be used in conjunction with any of the above aspects, the scoring member is configured to contact the target site when the balloon is in the inflated condition.

In some aspects, which can be used in conjunction with any of the above aspects, wherein the portion of the scoring member located in the channel when the sheath is in the delivery condition includes at least the proximal end region and the body portion of the scoring member.

In some aspects, which can be used in conjunction with any of the above aspects, wherein the distal end region is located distal to the channel when the sheath is in the delivery condition.

In some aspects, which can be used in conjunction with any of the above aspects, wherein at least the distal end region and the body portion are located distal to the channel when the sheath is in the scoring condition.

In some aspects, which can be used in conjunction with any of the above aspects, wherein the sheath is an elongated tube.

In some aspects, which can be used in conjunction with any of the above aspects, wherein the channel is a lumen formed in a wall of the elongated tube.

In some aspects, which can be used in conjunction with any of the above aspects, wherein the lumen is an elongate recess embedded in a wall of the elongated tube.

In some aspects, which can be used in conjunction with any of the above aspects, wherein the elongate recess is located in an interior surface of the sheath.

In some aspects, which can be used in conjunction with any of the above aspects, wherein the elongate recess is located in an exterior surface of the sheath.

In some aspects, which can be used in conjunction with any of the above aspects, wherein the elongate recess is a curved recess.

In some aspects, which can be used in conjunction with any of the above aspects, wherein the channel is included in a plurality of channels, and wherein the scoring member is included in a plurality of scoring members that is disposed in the plurality of channels.

In some aspects, which can be used in conjunction with any of the above aspects, wherein a quantity of the plurality of channels is equal to or greater than a quantity of the plurality of scoring members.

In some aspects, which can be used in conjunction with any of the above aspects, wherein the quantity of the plurality of scoring members is five or more.

In some aspects, which can be used in conjunction with any of the above aspects, wherein each of the plurality of scoring members are equidistantly spaced apart, are the same distance from a longitudinal axis of the sheathed scoring element, or both.

In some aspects, the present disclosure pertains to an apparatus for treatment of a vessel lesion, the apparatus comprising: a catheter shaft; a balloon having an exterior surface, wherein the balloon is configured to move between a deflated condition and an inflated condition; a sheathed scoring element that is separate from the balloon and includes: scoring members each having a proximal end region, a distal end region, and a body portion therebetween, wherein the at least the body portions of the scoring members are equidistantly spaced apart, are the same distance from a longitudinal axis of the sheathed scoring element, or both; and a sheath including a plurality of channels therein, wherein the sheath is configured to move relative to the scoring members between: a delivery condition where at least a portion of the each of the scoring members is located in the plurality of channels; and a scoring condition where the portion of each of the scoring members is located distal to a distal end of the plurality of channels and is configured to contact a target site when the balloon is in the inflated condition.

In some aspects, which can be used in conjunction with any of the above aspects, wherein each channel of the plurality of channels includes an individual scoring member of the plurality of scoring members.

In some aspects, which can be used in conjunction with any of the above aspects, wherein the scoring members are scoring wires.

In some aspects the present disclosure pertains to an apparatus for treatment of a vessel lesion, the apparatus comprising: a catheter shaft having a distal tip; a non-compliant balloon having a proximal end, a distal end, and a body portion having an exterior surface, wherein the balloon is configured to move between a deflated condition and an inflated condition; a sheathed scoring element that is separate from the balloon and includes: scoring wires each having a proximal end region, a distal end region, and a body portion therebetween, wherein the proximal end region of each of the scoring wires is coupled at least to the distal tip of the catheter shaft; and a sheath including a plurality of channels therein, wherein the sheath is configured to move relative to the scoring members between: a delivery condition where at least a portion of the each of the scoring wires is located in the plurality of channels; and a scoring condition where the portion of each of the scoring wires is located distal to a distal end of the plurality of channels and is configured to contact a target site when the balloon is in the inflated condition.

In some aspects, which can be used in conjunction with any of the above aspects, wherein the balloon is a non-complaint balloon.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

FIG. 1A is a simplified partial longitudinal view of an example catheter including an example of a sheathed scoring element in a delivery condition disposed in a blood vessel;

FIG. 1B is a simplified partial longitudinal view of an example catheter including an example of a sheathed scoring element in a scoring condition disposed in a blood vessel; FIG. 1C is a simplified partial longitudinal view of a system for treatment of a vessel lesion including the example catheter, the example sheathed scoring element in a scoring condition, and a balloon (a medical balloon) in an inflated condition disposed in the blood vessel;

FIG. 2A is a section view of the example of the sheathed scoring element in an example embodiment;

FIG. 2B is a section view of the example of the sheathed scoring element in another example embodiment;

FIG. 2C is a section view of the example of the sheathed scoring element in yet another example embodiment; and

FIG. 3 is a perspective view of an example sheathed scoring element.

### DETAILED DESCRIPTION

The following description should be read with reference to the drawings wherein like reference numerals indicate like elements throughout the several views. The detailed description and drawings illustrate example embodiments of the claimed invention.

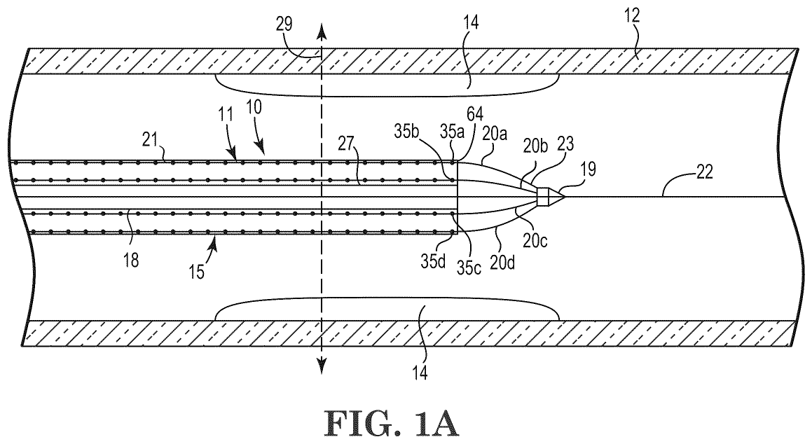

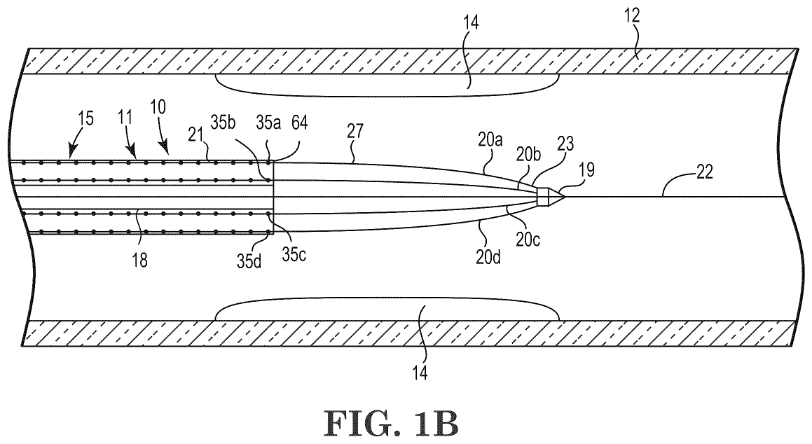

Angioplasty techniques have been shown to be effective for at least some intravascular interventions. FIG. 1A illustrates a simplified view of an example angioplasty catheter 10 including an example of a sheathed scoring element 11 positioned in a blood vessel 12 adjacent to a target site 14, namely an intravascular lesion or stenosis. FIG. 1B is a simplified partial longitudinal view of an example catheter 10 including an example of a sheathed scoring element 11 in a scoring condition disposed in a blood vessel. FIG. 1C is a simplified partial longitudinal view of a system for treatment of a vessel lesion including the example catheter 10, the example sheathed scoring element 11 in a scoring condition, and a balloon 16 (a medical balloon) in an inflated condition disposed in the blood vessel.

The target site 14 refers to stenosis or stricture, such as a hardened vessel lesion (e.g., a calcified lesion) or a soft plaque build-up (e.g., cholesterol), within the blood vessel 12. For instance, in some examples the target site 14 refers to hardened vessel lesion (e.g., a calcified lesion).

In some embodiments, the sheathed scoring element 11 is separate from the balloon (e.g., a balloon 16 as illustrated in FIG. 1C), unlike some other approaches such as those that employ a cutting balloon with a scoring member affixed to a surface (e.g., an exterior surface) of the balloon in a permanent manner. As used herein, having the components (e.g., the sheathed scoring element 11 and the balloon 16) be “separate” refers to the components not being coupled together (e.g., permanently coupled together) at least prior to and during delivery to a target site in a vessel. Having the sheathed scoring element 11 be separate from the balloon 16 can promote aspects herein. For instance, the systems and apparatuses herein can provide improved deliverability to, positioning at, and/or retention at a target site 14 in a blood vessel due at least in part to the sheathed scoring element 11 and the balloon 16 being separate components (e.g., rather than as an all-in-one device in which the balloon includes a scoring member permanently coupled thereto). That is, in various embodiments the balloon 16 herein does not include a scoring member (e.g., does not include a scoring member permanently affixed to an exterior surface of the balloon 16). Additionally, the system and apparatuses herein at least due in part to the sheathed scoring element 11 and the balloon 16 being separate components can include a larger quantity of scoring members (e.g., five or more scoring members, etc.) and/or can include scoring members that exhibit an improved scoring or cutting force at a target site 14 in a vessel. For instance, the system and apparatuses herein can permit the use of smaller dimension delivery devices (e.g., smaller catheters) as the individual components can have respective radial cross-sections that are smaller than corresponding radial cross-sections of all-in-one devices (e.g., a cutting balloon including a balloon and at least one scoring member permanently affixed thereto), can permit inclusion of a larger quantity of scoring members while still providing a suitable cross-section of the sheathed scoring element, and/or can permit the use of scoring elements that can provide a higher scoring or radial holding force at a target site in a vessel.

The sheathed scoring element 11 includes a sheath 15. The sheath 15 can be manifested as a tubular member, such as a circular, oval, or other shaped element having a substantially uniform cross-section (e.g., a substantially uniform radial and/or longitudinal cross-section). For instance, the sheath 15 can be an elongated tube. The sheath 15 can be formed of a polymer or metal, in some instances. For example, the sheath 15 can be formed of a metal such as stainless steel and/or another type of metal. However, in some embodiments, the sheath 15 can be a flexible sheath such as an elastomeric sheath.

The sheath 15 can have a smooth surface (e.g., an outer surface that is a radial outermost surface of the sheath and/or an inner surface defining a lumen of the sheath 15 and/or can have substantially uniform dimensions along a length of the sheath 15 to promote the sheath 15 to readily move (e.g., longitudinally translate) relative to the scoring member and/or the catheter 10 (e.g., the catheter shaft 18). That is, the sheath 15 can be a movable sheath that is configured to move in a substantially longitudinal direction relative at least to a scoring member in the sheathed scoring element 11. For example, the sheath 15 can be configured to move relative to each scoring member in the sheathed scoring element 11. In some embodiments, the sheath 15 can be configured to move relative to the scoring member between a first (e.g., delivery) condition where at least a portion of the scoring member is located in a channel of the sheath 15; and a second (e.g., scoring) condition where the portion of the scoring member is located distal to a distal end of the channel, and thus is exposed from the sheath 15 and is configured to contact a target site (e.g., when a balloon is in an inflated condition), as detailed herein. That is, the sheath 15 can initially be configured in the first (e.g., delivery condition) and can subsequently be moved (relative to the scoring member) to the second (e.g., scoring condition) in vivo. For instance, the sheath 15 can be moved from the delivery condition to the scoring condition in vivo when the sheathed scoring element 11 is positioned at or proximate to the target site 14. In some embodiments, the sheath 15 can be retracted proximally from the delivery condition to the scoring condition by actuation of a handle or other mechanism coupled to the sheathed scoring element 11 (e.g., coupled to a proximal end or proximal end region of the sheathed scoring element 11).

The sheath 15 can be configured to move relative to the scoring member 20 (e.g., scoring members 20a, 20b, 20c, and 20d, as illustrated in FIGS. 1A-1C) at least due to the presence of a channel 35 (e.g., channel 35a, 35b, 35c, and 35d as illustrated in FIGS. 1A-1C) formed in the sheath 15.

The channel can be manifested as one or more a substantially longitudinally extending channels that permit that sheath 15 to translate along the one or more scoring members. That is, the channel can be configured to receive at least a portion of a scoring member within the channel and permit the sheath 15 to move (e.g., longitudinally) relative to the scoring member that is at least partially located within the channel. For example, the channel can have a smooth surface (e.g., that is without any protrusions or recess therein) and/or can have uniform dimensions (e.g., a substantially uniform radial cross-section) along a length of the channel that can promote the channel (and thereby the sheath 15) to readily move relative to the scoring member.

In some embodiments, the channel can be included in a plurality of channels. In such embodiments, each of the channels can be the same size (e.g., having the same longitudinal length and diameter) and can extend in substantially the same direction within the sheath 15. For instance, each of the plurality of channels can be the same shape, the same size, and extend in substantially the same direction (e.g., be manifested as substantially longitudinally extending channels). In some embodiments, the channel can have a substantially circular or oval cross-section (e.g., radial cross-section), among other possible shapes. In some embodiments, the channel can have the same shape as the scoring member and/or can have a complimentary shape relative to the scoring member. For instance, in some embodiments the scoring member can have a round or circular cross-section (e.g., a radial cross-section), as detailed herein, and the channel can have a round or circular cross-section, among other possibilities. Employing channels and scoring members that have the same shape can promote aspects herein such as promoting ease of movement of the sheath 15 relative to the scoring members and/or mitigating any entanglement between the scoring members. However, in some embodiments, the channels can have a different shape (e.g., a C-shape or a U-shape, etc.) than a shape (e.g., a circular or round shape) of the scoring members, as detailed herein.

In some embodiments, the sheath 15 can be manifested as an elongated tube, as illustrated in FIGS. 1A-1C. The elongated tube can include one or more channels formed in a wall of the elongated tube and/or one or more channels manifested as elongate recesses in the wall (extending a radial distance into a surface of the wall) of the elongated tube. For instance, the sheath 15 can be an elongated tube that includes a channel manifested as a lumen formed in a wall (e.g., can be formed entirely within and enclosed by the wall) of the elongated tube. For example, the sheath 15 can be an elongated tube that only includes one or more channels manifested as one or more lumens formed in a wall of the elongated tube.

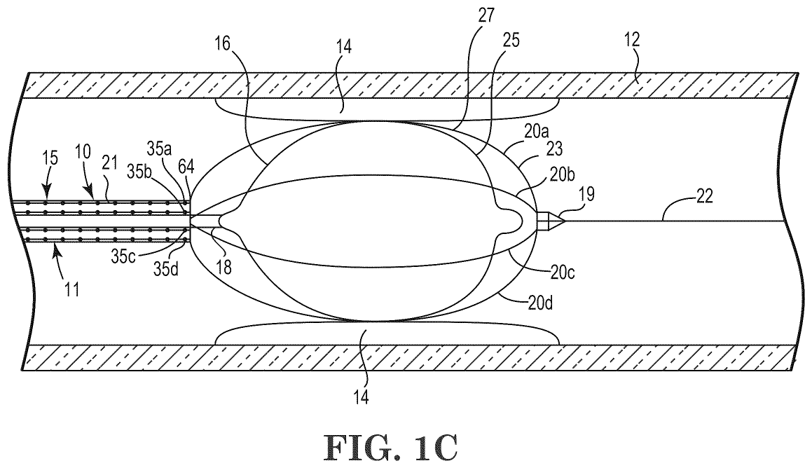

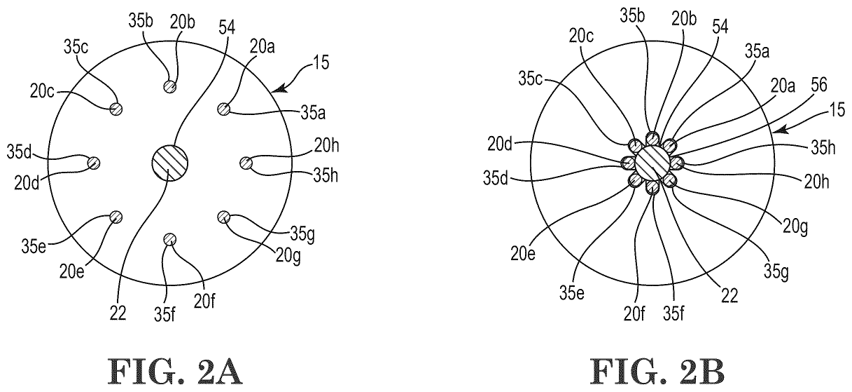

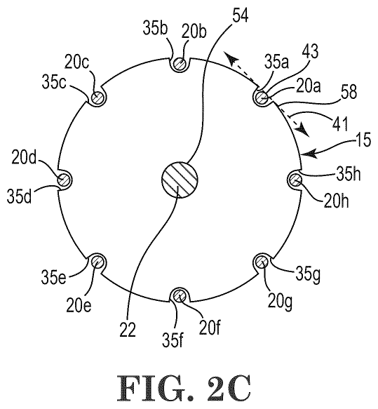

However, in some embodiments the sheath 15 can be an elongated tube that includes a channel manifested as an elongate recess in a wall of the elongated tube. The elongate recess can be present in an inner surface of the wall of the elongated tube and/or can be present in an outer surface of the elongated tube, as detailed herein with respect to FIGS. 2B-2C. That is, the elongate recess can extend a distance into a surface (e.g., the inner surface and/or the outer surface) of the elongated tube. Stated differently, the recess can extend a distance radially into the surface of the wall of the elongated tube but does not extend entirely (radially) through the wall, as described herein. For instance, in some embodiments the recess (e.g., an elongate recess) can be located in an inner surface of the sheath 15. For instance, the sheath 15 can include a plurality of recesses each formed circumferentially about an inner surface of the sheath (e.g., as illustrated in FIG. 2B). However, in some embodiments the recess (e.g., an elongate recess) can be located in an outer surface of the sheath 15. For instance, the sheath 15 can include a plurality of recesses each formed circumferentially about an outer surface of the sheath 15 (e.g., as illustrated in FIG. 2C).

In some embodiments, the sheath 15 can include a plurality of channels therein. For instance, the sheath 15 can include a plurality of channels formed in a wall of the sheath 15 that are each located the same respective distance from a longitudinal axis of the catheter shaft 18 and/or a longitudinal axis of the sheathed scoring element 11. In some embodiments, each of the plurality of channels can be equidistantly spaced apart. Stated differently, a distance between any channel and an immediately adjacent channel can be the same for each of the plurality of channels. Employing channels that are located at the same respective distance from a longitudinal axis of the catheter shaft 18 and/or the sheathed scoring element 11 and/or that are equidistantly spaced can promote aspects herein such as promoting the sheath to readily move relative to the scoring elements, mitigating any entanglement between the scoring elements, and/or providing uniform contact with and/or uniform cutting force on the target site 14 in a vessel. In some embodiments, each of the channels (e.g., 35a, 35b, 35c, and 35d, as illustrated in FIGS. 1A-1C) in the sheath 15 can be located the same respective distance from a longitudinal axis of the sheathed scoring element 11 and can be equidistantly spaced apart. Similarly, each of the scoring members (e.g., scoring members 20a, 20b, 20c, and 20d, as illustrated in FIGS. 1A-1C) in the sheath 15 can be equidistantly spaced apart, can be the same distance from a longitudinal axis of the sheathed scoring element 11, or both. For instance, each of the scoring members can be located the same respective distance from a longitudinal axis of the sheathed scoring element 11 and can be equidistantly spaced apart, as illustrated in FIGS. 2A-2C. For example, at least the body portions of the respective scoring members can be located the same respective distance from a longitudinal axis of the sheathed scoring element 11 and can be equidistantly spaced apart, as illustrated in FIGS. 2A-2C.

In some embodiments, each of the plurality of channels can have the same size and/or shape. For instance, each of the plurality of channels can be longitudinally extending channels that are the same shape and the same size (e.g., have the same longitudinal length). In some embodiments, each of the plurality of channels can be configured to receive a scoring member. For example, each of the plurality of channels can be configured to receive an individual scoring member. Stated differently, in some embodiments only a single scoring member is disposed within a respective channel of the plurality of channels. For instance, each of the channels 35a, 35b, 35c, and 35d can include at least a portion of the respective scoring members 20a, 20b, 20c, and 20d therein, as illustrated in FIGS. 1A-1C (e.g., the channels are represented as a series of longitudinally extending dots in FIGS. 1A-1C).

The sheathed scoring element 11 includes a scoring member (e.g., scoring members 20a, 20b, 20c, and 20d, as illustrated in FIGS. 1A-1C) and a sheath (e.g., sheath 15, as illustrated in FIGS. 1A-1C). The scoring member can extend substantially longitudinally (e.g., in a substantially linear, arched, or sinusoidal manner, etc.) along at least a portion along the catheter shaft 18. The scoring member can be a flexible scoring member that is configured to move radially (e.g., includes a body portion which is configured to expand radially) responsive to the application of force by a balloon (e.g., when in an inflated condition), as described herein. For instance, the scoring member can be a flexible wire, among other possibilities. For example, the scoring member 20 can be a scoring wire such as is illustrated with respect to the scoring members 20a, 20b, 20c, and 20d in FIGS. 1A-1C. The scoring wire can have a diameter in a range from about 0.2032 millimeters (0.008 inches) to about 0.3556 millimeters (0.014 inches). In some embodiments, the scoring wire can be a round elongate scoring wire having a substantially circular cross-section; however, other shapes such as those described herein are possible. The balloon 16 and the scoring member 20 (e.g., a rounded/circular or triangular scoring element, like a wire) can be easier to navigate (e.g., at least due to being provided as separate components that are delivered separately via the catheter 10 to the target site 14) and at least therefore yield less risk of vessel dissection/perforation, for instance as compared to commercially available cutting blades.

The scoring member can be manifested as an individual scoring member (e.g., formed of an individual wire or blade) or can be manifested as a plurality of scoring members. In embodiments, some or all of a plurality of scoring members can have the same shape and/or size. For example, the scoring members 20a, 20b, 20c, and 20d can each be wires that are the same shape and size, as illustrated in FIGS. 1A-1C.

In some embodiments, the apparatuses herein can include at least one pair of diametrically opposed scoring members (e.g., scoring wires) each having a proximal end, a distal end, and a body portion therebetween, where the body portion is not engaged with the catheter 10, and where, when the balloon 16 is in the inflated condition, the diametrically opposed scoring members are configured to contact diametrically opposed surfaces of the vessel lesion with a scoring force (e.g., at the same time).

In some embodiments, the scoring member can be manifested as an individual scoring member, two or more scoring members, three or more scoring members, four or more scoring members, five or more scoring members, six or more scoring members, seven or more scoring members, or eight or more scoring members. In some embodiments, the systems and apparatuses herein, at least due to providing the sheathed scoring element 11 separate from the balloon can permit inclusion of a greater quantity of scoring members than a quantity of scoring members (e.g., one to four scoring members) included in other types of medical devices such as those which include a balloon with one or more scoring members permanently affixed thereto. For instance, the sheathed scoring element 11 can include five or more scoring members, six or more scoring members, seven or more scoring members, eight or more scoring members, etc.

In some embodiments, a quantity of the channels (e.g., “N” channels) can be equal to or greater than a quantity of the scoring members. For instance, in some embodiments a quantity of the channels (e.g., “N” channels) can be equal to a quantity of the scoring members (e.g., “N”scoring members).

The scoring member can be a fixed scoring member. That is unlike the sheath 15 which can be configured to move relative to the catheter shaft 18 (e.g., is not permanently coupled to the catheter shaft 18), the scoring member can remain fixed to or permanently coupled to the catheter shaft 18. For instance, the scoring member can include at least one end or a portion thereof that is permanently coupled to the catheter 10 (e.g., coupled to the catheter shaft 18). For example, the scoring member can include a distal end region that is engaged (e.g., secured or affixed in a permanent fashion) to at least the distal tip of the catheter 10 and can extend proximally therefrom. The proximal end of the scoring member can be coupled to another portion of the catheter 10 (e.g., a handle or catheter shaft). In any case, at least a body portion of each of the scoring members 20a, 20b, 20c, and 20d can be configured to be engaged along an exterior of the balloon 16 when the balloon is present in a vessel, as detailed herein. That is, each of the scoring members can include a proximal end region, a distal end region, and a body portion. For example, the scoring member 20a can include a proximal end region 21 (shown in FIG. 1B), a distal end region 23, and a body portion 27. As mentioned and as illustrated in FIG. 1A, the distal end region 23 (e.g., a distalmost portion thereof) can be coupled to the distal tip 19 of the catheter 10 and the proximal end region 21 can be coupled to the catheter 10. The body portion 27 can remain unattached to the catheter 10 and thereby can permit movement of at least the body portion 27 relative to the catheter 10. The body portion 27 can move radially relative to the catheter 10 to permit the scoring member to radially expand (move away from the catheter 10) and contact the target site 14 when the balloon 16 is present and is an inflated configuration, as illustrated in FIG. 1C. In some embodiments, the distal end or distal end region 23 of scoring member 20 may be attached to the shaft 18 at a position distal of balloon 16, and a proximal end of scoring member 20 may be coupled to a more proximal portion of the catheter 10 (e.g., a handle or catheter shaft). However, this configuration is not intended to be limiting as it is contemplated that any part of scoring member 20 may be attached to shaft 18 at any suitable position. The type of attachment may also vary. For example, scoring member 20 may be attached to shaft 18 by welding, laser bonding, soldering, brazing, adhesive bonding, by using a mechanical fitting or connector, by winding or wrapping scoring member 20 around shaft 18, and the like, or in any other her suitable way.

The representative embodiments herein show the scoring members as substantially round wires (having a circular cross-section) extending substantially longitudinally manner. It is, however, contemplated that any or all of the scoring members may extend over any portion of the balloon in an alternative direction. For example, a scoring member may extend radially around the balloon. Alternatively, a scoring member may follow a generally helical pattern over the balloon.

Moreover, embodiments that include a plurality of scoring members may include a plurality of the same or similar scoring members or a combination of differing scoring member embodiments (e.g., with differing thicknesses, differing shapes, and/or differing orientations, etc.), including any of those described herein. In some embodiments, the scoring members may take a different shape such as a triangular shape where an apex of the triangle is configured to extend radially. In such instances, the triangular shaped scoring wire can reduce an amount of surface area of the scoring member (e.g., the apex of the triangle) in contact with a target site and thereby can provide a further improvement in an amount of scoring force imparted by the scoring wire on the target site. In such embodiments, a base side of the triangle can be positioned toward a longitudinal axis of the systems and apparatuses herein. Of course, other shapes can be used in various forms of scoring members without departing from the spirit of the disclosure. For example, various embodiments of scoring members may have circular, square, rectangular, polygonal, or any other suitable cross-sectional shape. The degree of curvature, pattern of curvature, and positioning of curves along the length of scoring members may also vary to include essentially any appropriate configuration.

It is further contemplated that the channels may extend through the sheath in the same direction, have different shapes, and/or different sizes. For instance, a direction, shape, quantity, and/or size of the channels herein may be altered in a similar manner as the scoring members (e.g., the direction of the representative channels may be altered to match or otherwise accommodate the shape, size, and/or direction of the scoring members).

Catheter 10 (e.g., an apparatus) may include a balloon 16 that is coupled thereto or configured to extend through a lumen of the catheter shaft 18. In some embodiments, the balloon 16, the sheathed scoring element 11, and/or the catheter 10 may be manufactured from a lubricious material. Alternatively, the balloon 16, the sheathed scoring element 11, and/or catheter 10 may be coated with a lubricious material. Lubricity may be desirable for a number of reasons, such as to enhance the ability of the balloon 16 and/or the sheathed scoring element 11, to be navigated through the vasculature, particularly when advancing catheter 10 through a relatively narrow or occluded vessel and to minimize friction against ancillary devices such as guide catheters. The at least one scoring member 20 can contact a target site and thereby permit scoring a lesion, when the balloon 16 is in an inflated condition, as described herein. For instance, at least the body portions of each of the scoring members (e.g., each of 20a, 20b, 20c, and 20d, as illustrated in FIGS. 1A-1C) can be configured to expand radially outward responsive to contact with a respective portion of an exterior surface 25 (FIG. 1C) of the balloon 16, thereby providing a uniform or improved scoring force at each of the portions (e.g., body portions) of the scoring members 20 that contacts a target site. For example, scoring member 20 can provide a surface that can help maintain the position of balloon 16 (e.g., keep balloon 16 from slipping away from target site 14), can provide an improved scoring force, and/or may allow target site 14 (hardened lesion, soft plaque) to be expanded more precisely and prepare the lesion site for stenting, if scaffolding is needed, facilitating correct stent expansion. Moreover, the balloon 16 and the sheathed scoring element 11 herein do not require or include the hinge points such as a hinge point that may typically be employed on commercially available cutting blades or atherotomes and which can partially detach the atherotome/blade from the balloon particularly in tortuous vasculature. Additionally, the balloon 16 and/or the sheathed scoring element 11 including a sheath 15 and a scoring member 20, as described herein, can be shorter in length (a longitudinal length) and/or the balloon 16 does not require folding. For these reasons the balloon 16 and the sheathed scoring element 11 can be comparatively easier to navigate (e.g., as the balloon 16 can conform to a bend in a vessel) and yet, in some embodiments can also permit a drug coating to de-bond from the balloon 16 and readily transfer to a target site (e.g., vessel wall) when the balloon 16 is inflated (e.g., is in an inflated configuration). The balloon 16 and/or the sheathed scoring element 11 can also permit drug transfer to a target site (e.g., arterial wall) by inducing therapeutic dissections at the same time as drug transfer takes place. Some of the other features, characteristics, and alternative embodiments of the scoring member 20 are described in more detail below.

Balloon 16 can be a compliant, non-compliant, or semi-compliant balloon. For instance, in some embodiments the balloon 16 can be a semi-compliant balloon or non-compliant balloon. In some embodiments that balloon 16 can be a non-compliant balloon. Employing a non-compliant balloon can promote aspects herein such as promoting the radial expansion of at least a portion (e.g., a body portion) of a scoring member and thereby causing the scoring member to contact or score a target site 14.

In various embodiments, the balloon 16 can be a substantially cylindrical or spherical balloon, among other possible shapes. For instance, the balloon can have a have a radial cross-section (e.g., as taken at the widest radial portion or cross-section thereof) in a range from about 2.5 millimeters (0.098 inches) to about 5.7 millimeters (0.224 inches) or from about 2.5 millimeters (0.098 inches) to about 4.5 millimeters (0.177 inches), when the balloon 16 is in an inflated condition. Other values of the radial cross-sections of the balloon 16 are possible, and may be varied, for instance, to match the diameters of the different vessels in the vasculature.

The balloon 16 can be made from typical angioplasty balloon materials including polymers such as polyethylene terephthalate (PET), polyetherimide (PEI), polyethylene (PE), etc. Some other examples of suitable polymers, including lubricious polymers, may include polytetrafluoroethylene (PTFE), ethylene tetrafluoroethylene (ETFE), fluorinated ethylene propylene (FEP), polyoxymethylene (POM), polybutylene terephthalate (PBT), polyether block ester, polyurethane, polypropylene (PP), polyvinylchloride (PVC), polyether-ester (for example, a polyether-ester elastomer such as ARNITEL® available from DSM Engineering Plastics), polyester (for example, a polyester elastomer such as HYTREL® available from DuPont), polyamide (for example, DURETHAN® available from Bayer or CRISTAMID® available from Elf Atochem), elastomeric polyamides, block polyamide/ethers, polyether block amide (PEBA, for example, available under the trade name PEBAX®), silicones, MARLEX® high-density polyethylene, MARLEX® low-density polyethylene, linear low density polyethylene (for example, REXELL®), polyetheretherketone (PEEK), polyimide (PI), polyphenylene sulfide (PPS), polyphenylene oxide (PPO), polysulfone, nylon, perfluoro(propyl vinyl ether) (PFA), other suitable materials, or mixtures, combinations, copolymers thereof, polymer/metal composites, and the like. In some embodiments, it may be desirable to use high modulus or generally stiffer materials so as to reduce balloon elongation. The above list of materials includes some examples of higher modulus materials. Some other examples of stiffer materials include polymers blended with liquid crystal polymer (LCP) as well as the materials listed above. For example, the mixture can contain up to about 5% LCP. Alternatively, the balloon 16 may be coated with a relatively lubricious material such as a hydrogel or silicone.

In general, catheter 10 may be advanced over a guidewire 22 through the vasculature to a target site 14. The sheathed scoring element 11 can then be advanced over the guidewire 22 through the vasculature to the target site. Prior to and during advancement of the sheathed scoring element 11 the sheath 15 can cover or overlay at least a portion of a scoring member of the sheathed scoring element 11. For instance, the sheath 15 can include one or more channels in which at least a portion of one or more scoring members 20 are disposed, as detailed herein. Once at the target site, the sheath 15 of the sheathed scoring element 11 can be moved (e.g., retracted proximally) to expose a portion of a scoring member of the sheathed scoring element 11 that was covered prior to and during advancement of the sheathed scoring element 11 to the target site. Subsequent to delivery of the sheathed scoring element 11 to the target site 14, the balloon 16 can be advanced (e.g., over the guidewire 22) through the vasculature to the target site 14. Once at the target site, the balloon 16 can then be inflated (e.g., to an inflated configuration) such that the exterior surface 25 of the balloon 16 contacts and radially expands the scoring member to cause the scoring member to cut/score and expand a target site 14 such as a lesion. The target site may be within any suitable peripheral or cardiac location, for example.

Shaft 18 may be a catheter shaft, similar to typical catheter shafts which have a distal end portion and a proximal end portion. For example, shaft 18 may include an inner tubular member (e.g., defining a guidewire lumen to accommodate a guide wire and track over it to the target lesion) and outer tubular member (e.g., defining an inflation lumen to allow passage of inflation media, usually contrast dye diluted with saline solution). Tubular members may be manufactured from a number of different materials. For example, tubular members may be made of metals, metal alloys, polymers, metal-polymer composites or any other suitable materials. Some examples of suitable metals and metal alloys include stainless steel, such as 304V, 304L and 316L stainless steel; nickel-titanium alloy such as linear-elastic or super-elastic Nitinol, nickel-chromium alloy, nickel-chromium-iron alloy, cobalt alloy, tungsten or tungsten alloys, MP35-N (having a composition of about 35% Ni, 35% Co, 20% Cr, 9.75% Mo, a maximum 1% Fe, a maximum 1% Ti, a maximum 0.25% C, a maximum 0.15% Mn, and a maximum 0.15% Si), Hastelloy, Monel 400, Inconel 825, or the like; or other suitable material. Some examples of suitable polymers include those described above in relation to balloon 16. Of course, any other suitable polymer may be used without departing from the spirit of the disclosure. The materials used to manufacture inner tubular member may be the same as or be different from the materials used to manufacture outer tubular member.

Tubular members may be arranged in any appropriate way. For example, in some embodiments inner tubular member can be disposed coaxially within outer tubular member. According to these embodiments, inner and outer tubular members may or may not be secured to one another along the general longitudinal axis of shaft 18. Alternatively, inner tubular member may follow the inner wall or otherwise be disposed adjacent the inner wall of outer tubular member. Again, inner and outer tubular members may or may not be secured to one another. For example, inner and outer tubular members may be bonded, welded (including tack welding or any other welding technique), or otherwise secured at a bond point. In some embodiments, the bond point may be generally disposed near the distal end portion of shaft 18 or near a proximal end portion of the shaft 18. However, one or more bond points may be disposed at any position along shaft 18. The bond may desirably impact, for example, the stability and the ability of tubular members to maintain their position relative to one another. In still other embodiments, inner and outer tubular member may be adjacent to and substantially parallel to one another so that they are non-overlapping. In these embodiments, shaft 18 may include an outer sheath that is disposed over tubular members.

Inner tubular member includes an inner lumen. In a preferred embodiment, inner lumen is a guidewire lumen. Accordingly, catheter 10 can be advanced over guidewire 22 to the desired location. The guidewire lumen may extend along essentially the entire length of catheter shaft so that catheter 10 resembles traditional “over-the-wire” catheters. Alternatively, the guidewire lumen may extend along only a portion of shaft 18 so that catheter 10 resembles “single-operator-exchange” or “rapid-exchange” catheters. Regardless of which type of catheter is contemplated, catheter 10 may be configured so that balloon 16 is disposed over at least a region of inner lumen or is configured to be advanced therethrough. For instance, the balloon 16 may be advanced through the guidewire lumen, through a different lumen (other than the guidewire lumen) in the catheter 10, or may be advanced separately from the catheter 10 (e.g., via a separate balloon catheter).

An inflation lumen can be in fluid communication with the balloon 16. For instance, shaft 18 may also include an inflation lumen in fluid communication with the interior of the balloon 16, that may be used, for example, to transport inflation media to and from balloon 16. For example, when an outer tubular member is disposed over an inner tubular member, the inflation lumen may be defined within the space between the outer and inner tubular members. Alternatively, the inflation lumen can be provided in a separate catheter shaft, for instance when the balloon is delivered via a catheter that is separate from the catheter 10. In some embodiments, the balloon 16 can be manifested as a single balloon 16. In some embodiments, the inflation lumen can be manifested as a single inflation lumen. In some embodiments, the balloon 16 can be a single balloon and the inflation lumen can be manifested as a single inflation lumen in fluid communication with an interior of the balloon 16. Thus, inflation media delivered through the inflation lumen may be introduced into the interior of the balloon 16 to inflate the balloon 16 (e.g., cause the balloon to shift from a deflated condition to an inflated condition). The balloon 16 can be inflated to a pressure (e.g., about 1600 kilopascals, etc.) in an inflated condition that is typically associated with inflatable balloons, for example.

As mentioned, the exterior surface 25 of the balloon 16 is configured to engage the scoring member 20 to apply a radially outward force against the scoring member 20 when the balloon 16 is inflated, which in turn exerts a scoring force against the target site 14. For instance, as illustrated in FIG. 1C, the balloon 16 is in an inflated configuration. While FIG. 1C illustrates the balloon in an inflated condition, in some embodiments the balloon 16 may have the same or similar shape and configuration (albeit with a different size) when the balloon 16 is in its nominal or deflated condition.

In addition to some of the structures described above, the catheter shaft 18 may also include a number of other structural elements, including those typically associated with catheter shafts. For example, the catheter shaft 18 may include a radiopaque marker coupled thereto that may aid a user in determining the location of catheter 10, such as the exterior surface 25 of the balloon 16 within the vasculature. In addition, catheter 10 may include a folding spring (not shown) coupled to balloon 16 which may further help in balloon folding and refolding. A description of a suitable folding spring can be found in U.S. Pat. Nos. 6,425,882 and 6,623,451, the disclosures of which are incorporated herein by reference.

In some embodiments, a drug-eluting sleeve (not shown) or therapeutic (e.g., drug-eluting) coating (not shown) may overlay at least a portion of a body (e.g., exterior surface) of the balloon 16. The drug-eluting sleeve or therapeutic coating may be coupled to catheter shaft 18 and/or the balloon 16 in any of a number of suitable ways. For example, the drug-eluting sleeve may be adhesively or thermally bonded to shaft 18, for instance, at locations along the catheter shaft 18 that are proximal and distal to the balloon 16. The drug-eluting sleeve may be configured (e.g., with an appropriate radial thickness and/or with an appropriate excipient) to permit drug-delivery prior to, during, and/or subsequent to scoring of a lesion with the scoring members herein. For instance, the drug-eluting sleeve can be configured to permit drug-delivery to a target site at least while scoring the target site with the scoring member, when the balloon is in an inflated condition. The presence of the drug-eluting sleeve or therapeutic coating at least on the balloon 16 can permit drug delivery prior to, during, and/or after scoring a lesion (e.g., a hardened lesion) with a scoring member. For instance, the example catheters employing the balloon 16 and the sheathed scoring element 11 as separate components can be configured to provide drug-delivery to a target site at the same time as the scoring members in the sheathed scoring element 11 score the target site (e.g., a hardened lesion). Providing drug-delivery to the target site at least at the same time as the scoring members score the target site can promote aspects herein such as mitigating inflammation associated with scoring the target site, thereby reducing a likelihood of the target site forming a new lesion and/or otherwise mitigating any unintended side-effects associated with scoring the target site. The scoring can induce therapeutic dissections on the vessel wall to enhance drug transfer and drug retention in the treated vessel wall.

In some embodiments, the drug-eluting sleeve or therapeutic coating on the surface of the balloon can include an excipient, an active agent and/or drug (e.g., an amorphous form of a drug or a crystalline form of a drug). Some specific beneficial agents include anti-thrombotic agents, anti-proliferative agents, anti-inflammatory agents, anti-migratory agents, pro-endothelization agents and/or other agents affecting extracellular matrix production and organization, antineoplastic agents, anti-mitotic agents, anesthetic agents, anti-coagulants, vascular cell growth promoters, vascular cell growth inhibitors, cholesterol-lowering agents, vasodilating agents, and agents that interfere with endogenous vasoactive mechanisms.

More specific drugs or therapeutic agents include paclitaxel, rapamycin, sirolimus, everolimus, tacrolimus, heparin, diclofenac, aspirin, Epo D, dexamethasone, estradiol, halofuginone, cilostazol, geldanamycin, ABT-578 (Abbott Laboratories), trapidil, liprostin, Actinomycin D, Resten-NG, Ap-17, abciximab, clopidogrel, Ridogrel, beta-blockers, bARKct inhibitors, phospholamban inhibitors, and SERCA 2 gene/protein, resiquimod, imiquimod (as well as other imidazoquinoline immune response modifiers), human apolipoproteins (e.g., AI, AII, AIII, AIV, AV, etc.), vascular endothelial growth factors (e.g., VEGF-2), as well as derivatives of the forgoing, among many others. In some embodiments, the drug may be a macrolide immunosuppressive (limus) drug. Other drugs may include anti-inflammatory agents such as dexamethasone, prednisolone, corticosterone, budesonide, estrogen, sulfasalazine, mesalamine, and analogues thereof; antineoplastic/antiproliferative/anti-miotic agents such as paclitaxel, 5-fluorouracil, cisplatin, vinblastine, vincristine, epothilones, endostatin, angiostatin, thymidine kinase inhibitors, and analogues thereof; anesthetic agents such as lidocaine, bupivacaine, ropivacaine, and analogues thereof; anti-coagulants; and growth factors.

In some instances, the drug-eluting sleeve or therapeutic coating may include individual drug particles that are encapsulated with one or more excipients. The drug particles may include crystals of the drug, for example. Drug crystals may be formed in a variety of ways, for example. In some cases, a drug or other therapeutic agent may be available in an amorphous form, and a variety of processes may be used to convert an amorphous drug or other therapeutic agent into a crystalline drug or other therapeutic agent.

In some instances, a drug-eluting sleeve can be disposed on (overlay and be in contact with) a balloon of the medical apparatuses (e.g., medical devices) herein. For example, a drug-eluting sleeve can be slid over a balloon when the balloon is in a deflated condition and can be bonded (e.g., via thermal bonding or adhesive bonding to portions of the balloon (e.g., at a proximal waist and a distal waist of the balloon). However, in some instances, the medical device may be contacted with the coating composition in order to form a coating on the medical device. In some instances, the medical device or a portion thereof may be dipped into the coating composition. In some cases, vapor deposition may be used to transfer the coating composition to the medical device. In some cases, a roller coating process may be used to transfer the coating composition/formulation to the medical device. In some cases, ink-jet printing technology may be used to apply the coating composition/formulation to the medical device. These are just examples. In some cases, the coating composition may be sprayed onto the medical device, or may be sprayed onto a particular portion or region of the medical device.

In some instances, one or more excipients may be employed. For instance, a mixture of excipients may be employed that includes two or more different excipients. An example excipient may include ethyl cellulose (EC), which is a derivative of cellulose in which some of the hydroxyl groups on the repeating glucose units are converted into ethyl ether groups. The relative number of ethyl ether groups can vary depending on the particular manufacturer. Another example excipient may include acetyl tri-butyl citrate (ATBC), which in some cases may be referred to by its IUPAC name of tributyl 2-acetyloxypropane-1,2,3-tricarboxylate.

In some cases, the mixture of excipients may optionally include one or more additional excipients, to ensure that the active pharmaceutical ingredient (drug) is retained by the coated sleeve while the catheter navigates the vasculature and transferred to the lesion upon balloon inflation (which stresses the elastic sleeve, releasing the drug).

FIGS. 2A-2C illustrate alternative section views (taken along section line 29 in FIG. 1A) of embodiments of various sheathed scoring elements. As illustrated in FIGS. 2A-2C each of the channels can be off-set (spaced radially) from a longitudinal axis of the sheathed scoring element 11.

FIG. 2A is a section view of the example of the sheathed scoring element where each of the channels 35a, 35b, 35c, 35d, 35e, 35f, 35g, 35h are manifested as a lumen formed in a wall 52 of the sheath 15. As illustrated in FIG. 2A, each of the lumens can be entirely embedded within (surrounded entirely by) the wall 52. Having the lumens be entirely embedded within the wall 52 can promote aspects herein such as mitigating any entanglement between the scoring members and/or promoting movement of the sheath 15 relative to the scoring members. As illustrated in FIG. 2A each of the channels 35a, 35b, 35c, 35d, 35e, 35f, 35g, 35h (collectively referred to herein as channels 35) can include at least a portion (e.g., a body portion) of a respective (individual) scoring member of a plurality of scoring members 20a, 20b, 20c, 20d, 20e, 20f, 20g, 20h (collectively referred to herein as scoring members 20) disposed therein. As illustrated in FIG. 2A, the channels 35 and the scoring members 20 can have substantially the same shape (e.g., can each have circular or round radial cross-sections) along a portion or an entire length thereof. As mentioned, employing channels 35 and scoring members 20 with the same shape can promote aspects herein such as promoting movement of the sheath 15 relative to the channels and/or mitigating any entanglement of the scoring members 20.

While FIG. 2A illustrates the channels as rounded or circular channels, in some embodiments the channels can have a different shape (e.g., a C-shape or U-shape, etc.). For instance, FIGS. 2B-2C are section views of the examples of sheathed scoring elements where the channels are manifested as curved or arcuate (e.g., U-shaped) recesses. That is, each of the channels can be manifested as curved channels such as a those formed of C-shaped or U-shaped recess. However, the recesses may have a different shape. In some embodiments each of the channels 35 can be manifested as recesses that are the same shape and recessed the same distance in a surface (e.g., an interior surface 56 or exterior surface 58) of the sheath, as illustrated in FIGS. 2B-2C. Employing recesses that are the same shape and are recessed the same distance (e.g., radially relative to a surface in which the recess is formed) can promote aspects herein such as promoting ease of movement of the sheath 15, etc. However, in some embodiments, one or more of the recesses can be a different shape or a different size.

FIG. 2B is analogous to FIG. 2A, with the change that the channels are manifested as recesses formed in the interior surface 56 of the sheath 15. That is, the recesses are present only in the interior surface 56 of the sheath 15, as illustrated in FIG. 2B. The interior surface 56 can correspond to a surface that defines a guidewire lumen 54. For instance, a guidewire such as the guidewire 22 can be disposed in the guidewire lumen 54, as illustrated in FIG. 2B. Each of the channels can be in fluidic communication with or are otherwise open to and coupled to the guidewire lumen, as illustrated in FIG. 2B. Thus, each of the scoring members 20 can be in physical contact with a portion of the guidewire 22, as illustrated in FIG. 2B.

FIG. 2C is analogous to FIG. 2B, with the change that the channels 35 are manifested as recesses formed in the exterior surface 58 of the sheath 15. That is, the recesses are present only in the exterior surface 58 of the sheath 15, as illustrated in FIG. 2C. As illustrated in FIG. 2C, the scoring members 20 can be disposed within the channels 35 (e.g., recesses) in the exterior surface 58 of the sheath 15. An outer surface (a radial most surface relative to a longitudinal axis of the sheath 15) of the scoring members 20 can be recessed a distance from the exterior surface 58 or can be coplanar with the exterior surface. For instance, the outer surface of each of the scoring members 20 can be coplanar with a portion of the exterior surface 58 that is adjacent to the channels 35 in which the scoring members 20 are disposed, as illustrated in FIG. 2C. For example, an outer surface 43 of the scoring member 20a can be coplanar (as represented by axis 41) with a portion of the exterior surface 58 of the sheath 15 that is adjacent to the channel 35a (e.g., recess), as illustrated in FIG. 2C.

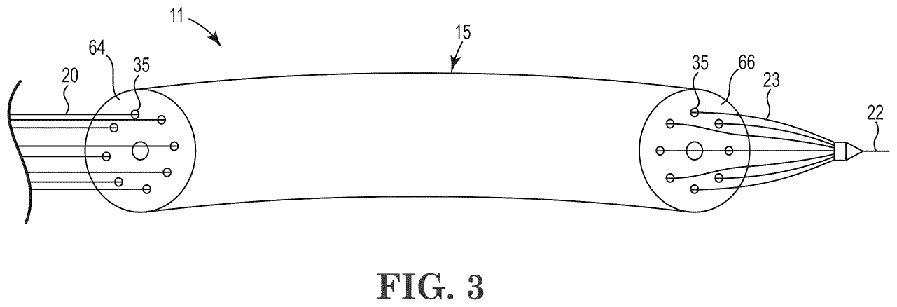

FIG. 3 is a perspective view of an example sheathed scoring element 11 including a sheath 15 and a plurality of scoring members 20. As illustrated in FIG. 3, the sheath 15 can be an elongated tube having a substantially planar distal end 64 and a substantially planar proximal end 66. The channels 35 in the sheath 15 can extend longitudinally in a substantially linear manner between the distal end 64 and the proximal end 66 of the sheath 15, as illustrated in FIG. 3. That is, the channels 35 can extend along an entire length of the sheath 15. Employing sheathed scoring elements with the channels 35 extending between the distal and proximal ends of the sheath 15 can promote aspects herein such as promoting movement of the sheath relative to at least a portion (e.g., a body portion) of the scoring members 20 and/or mitigating any entanglement of the scoring members 20 that may otherwise occur (e.g., in the absence of the channels).

In some embodiments, a distal end region of the scoring member can remain exposed when the sheathed scoring element and the scoring member are in a delivery configuration. For instance, as illustrated in FIG. 3, the distal end region 23 of the scoring member is exposed when the sheathed scoring element 11 and the scoring member 20 are in the delivery configuration. Stated differently, the distal end region 23 can be located distal to the distal end of 64 of the sheath 15 when the sheathed scoring element 11 and the scoring member 20 are in the delivery configuration. Yet, the body portion (not illustrated in FIG. 3) and the proximal end region (not illustrated in FIG. 3) of the scoring member 20 can be covered by the sheath 15 when the sheathed scoring element 11 and the scoring member 20 are in the delivery configuration. In some embodiments, the distal end region 23 can be a tapered distal end region having a larger radial cross-section at a proximal end thereof than a radial cross-section at a distal end thereof, as illustrated in FIG. 3. Having the distal end region 23 be exposed and the body portion and proximal end region be covered by the sheath 15 in the delivery configuration can promote aspects herein. For instance, the distal tip 19 and the exposed distal end region 23 can initially contact (e.g., form an opening in and/or radially expand) a target site, particularly when the distal end region 23 is manifested as a tapered distal end region. Yet, having the body portion and the proximal end region of the scoring member remain covered by the sheath 15 (e.g., be disposed in the channels of the sheath) can mitigate any inadvertent contact with tissue near the target site and/or can mitigate any entanglement of respective scoring members. Moreover, at least the body portion and the distal end portion of the scoring member can be exposed (e.g., are distal to a distal end region of the sheath) when the sheathed scoring element and scoring member are in the scoring condition, and thereby can permit at least the body portion of the scoring member to be radially expanded by an exterior surface of the balloon and contact (e.g., score) a target site, as detailed herein.

In at least some embodiments, portions or all of the medical apparatuses (devices) described herein may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the medical devices described herein in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the medical devices described herein to achieve the same result.

In some embodiments, a degree of Magnetic Resonance Imaging (MRI) compatibility is imparted into the medical apparatuses described herein. For example, the medical apparatuses described herein, or portions thereof, may be made of a material that does not substantially distort the image and create substantial artifacts (i.e., gaps in the image). Certain ferromagnetic materials, for example, may not be suitable because they may create artifacts in an MRI image. The medical devices described herein, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made to various details herein, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A system for treatment of a vessel lesion, the system comprising:
a catheter shaft;
a balloon having an exterior surface, wherein the balloon is configured to move between a deflated condition and an inflated condition; and
a sheathed scoring element that is separate from the balloon and includes:
a scoring member having a proximal end region, a distal end region, and a body portion therebetween; and
a sheath including a channel, wherein the sheath is configured to move relative to the scoring member between:
a delivery condition where at least a portion of the scoring member is located in the channel; and
a scoring condition where the portion of the scoring member is located distal to a distal end of the channel and is configured to contact a target site.

2. The system of claim 1, wherein the scoring member is configured to contact the target site when the balloon is in the inflated condition.

3. The system of claim 1, wherein the portion of the scoring member located in the channel when the sheath is in the delivery condition includes at least the proximal end region and the body portion of the scoring member.

4. The system of claim 3, wherein the distal end region is located distal to the channel when the sheath is in the delivery condition.

5. The system of claim 1, wherein at least the distal end region and the body portion are located distal to the channel when the sheath is in the scoring condition.

6. The system of claim 1, wherein the sheath is an elongated tube.

7. The system of claim 6, wherein the channel is a lumen formed in a wall of the elongated tube.

8. The system of claim 7, wherein the lumen is an elongate recess embedded in a wall of the elongated tube.

9. The system of claim 8, wherein the elongate recess is located in an interior surface of the sheath.

10. The system of claim 8, wherein the elongate recess is located in an exterior surface of the sheath.

11. The system of claim 8, wherein the elongate recess is a curved recess.

12. The system of claim 1, wherein the channel is included in a plurality of channels, and wherein the scoring member is included in a plurality of scoring members that is disposed in the plurality of channels.

13. The system of claim 12, wherein a quantity of the plurality of channels is equal to or greater than a quantity of the plurality of scoring members.

14. The system of claim 13, wherein the quantity of the plurality of scoring members is five or more.

15. The system of claim 12, wherein each of the plurality of scoring members are equidistantly spaced apart, are the same distance from a longitudinal axis of the sheathed scoring element, or both.

16. An apparatus for treatment of a vessel lesion, the apparatus comprising:
a catheter shaft;
a balloon having an exterior surface, wherein the balloon is configured to move between a deflated condition and an inflated condition;
a sheathed scoring element that is separate from the balloon and includes:
scoring members each having a proximal end region, a distal end region, and a body portion therebetween, wherein the at least the body portions of the scoring members are equidistantly spaced apart, are the same distance from a longitudinal axis of the sheathed scoring element, or both; and
a sheath including a plurality of channels therein, wherein the sheath is configured to move relative to the scoring members between:
a delivery condition where at least a portion of the each of the scoring members is located in the plurality of channels; and
a scoring condition where the portion of each of the scoring members is located distal to a distal end of the plurality of channels and is configured to contact a target site when the balloon is in the inflated condition.

17. The apparatus of claim 16, wherein each channel of the plurality of channels includes an individual scoring member of the plurality of scoring members.

18. The apparatus of claim 16, wherein the scoring members are scoring wires.

19. An apparatus for treatment of a vessel lesion, the apparatus comprising:
a catheter shaft having a distal tip;
a non-compliant balloon having a proximal end, a distal end, and a body portion having an exterior surface, wherein the balloon is configured to move between a deflated condition and an inflated condition;
a sheathed scoring element that is separate from the balloon and includes:
scoring wires each having a proximal end region, a distal end region, and a body portion therebetween, wherein the proximal end region of each of the scoring wires is coupled at least to the distal tip of the catheter shaft; and
a sheath including a plurality of channels therein, wherein the sheath is configured to move relative to the scoring members between:
a delivery condition where at least a portion of the each of the scoring wires is located in the plurality of channels; and
a scoring condition where the portion of each of the scoring wires is located distal to a distal end of the plurality of channels and is configured to contact a target site when the balloon is in the inflated condition.

20. The apparatus of claim 19, wherein the balloon is a non-complaint balloon.
